(19) United States
(12) Patent Application Publication
(10) Pub. No.: US 2005/0112557 A1
(43) Pub. Date: May 26, 2005

(54) Mesoporous-chip based biosensor for rapid biological agent detection

(76) Inventors: Yongcheng Liu, Fayetteville, AR (US); Yixiang Duan, Los Alamos, NM (US)

Correspondence Address: Yixiang Duan, 50 Lantana Dr., Los Alamos, NM, 87544, US

(21) Appl. No.: 10/947,099

(22) Filed: Sep. 22, 2004

Related U.S. Application Data
(60) Provisional application No. 60/505,398, filed on Sep. 25, 2003.

(51) Int. Cl. C12Q 1/70; G01N 33/554; G01N 33/569; C12M 1/34

(52) U.S. Cl. 435/5; 435/287.2; 356/319; 435/7.32

(57) ABSTRACT

The present invention includes a new, sensitive, rapid, portable, and inexpensive biosensor for detection of biological agents. The inventors develop a mesoporous-chip based biosensor device that is able to detect very low-level pathogens in a relatively short time. This biosensor device is designed in a way that significantly increases the reaction area, and constructed by immobilizing antibodies onto a mesoporous chip surface. The antibody-immobilized mesoporous chip is used as a bioseparator for separation of pathogens or other biological agents when the sample goes through the chip pores. Then an enzyme labeled antibody solution is injected into the chip pores, and a sandwich structure of immuno-complexes (enzyme labeled antibody-biological agent-antibody immobilized on chip) can be formed within the chip pores. The porous chip will also be a bioreactor for catalysis of the enzyme reaction, resulting in easily detected chemical species. The pathogens or other biological agents can be detected through measuring the absorbance or fluorescence of the enzyme reaction and its products. The dramatic increase of the reaction/surface area in the mesoporous chip significantly increases the sensitivity of the biosensor device and shortens the detection time.

### FIELD OF THE INVENTION

The present invention relates generally to an apparatus and accompanying method for detection of biological agents. More particularly, the apparatus and method utilizes a mesoporous-chip based optical biosensor and enzyme reaction for the detection of biological agents.

### BACKGROUND OF THE INVENTION AND RELATED ARTS

Microbial contamination is a major concern of the food industry. Part of the challenge that faces the food industry charged with protecting public health is to find more effective and rapid technology to detect specific pathogens, such as Salmonella typhimurium, Campylobacter jejune, Listeria monocytogenes, and Escherichia coliO157:H7, before food products are distributed to the public. The Center for Disease Control and Prevention (CDC) estimated that contaminated food made 76 million people sick, more than 300,000 people hospitalized, and about 5,000 Americans dead in the Untied States each year. The annually economic impact of foodborne illness has been estimated as high as $10 billions. The foodborne pathogenic contamination of food products also incurred a heavily economic burden for the industry due to product recalls. In addition, the detection of pathogens in environment is also extremely important. For example, the annual loss caused by biological fouling and microbially induced corrosion has been estimated to be billions of U.S. dollars in industrial water handling systems. The threat from biological warfare and bioterrorism has also become a critical issue both in the battlefield and for the general public. Bacteria are considered to be potentially the most prevalent type of biological warfare agent among the many classes of biological warfare agents available because they are robust, easy to deliver, and result in acute or delayed toxicity. After Sep. 11, 2001, terrorists' Anthrax mails caused huge economic loss in the United States and several fatal incidents were reported.

The conventional cultural method for detection of bacteria typically needs 3-7 days to confirm the analysis if presumptive positive bacterial colonies develop. Therefore, there is a critical need for establishing rapid and reliable analytical techniques for fast detection of these low concentration pathogens in the food industries, supermarkets, wastewater treatment plants, door air, potable drinking water, fossil, and nuclear power plants, as well as battlefields.

Biosensor technology has been actively explored in recent years. The technology shows possibility of rapid detection of pathogens through direct and/or on-line testing for fast food-safety assessment. Many types of biosensors have been developed to detect the pathogens. The piezoelectric biosensors were developed for rapid detection of Candida albicans in a range from 10⁶ to 5×10⁸ cells/ml, S. typhimurium from 10⁵ to 10⁹ CFU/ml within 5 h analysis and from 9.9×10⁵ to 1.8×10⁸ CFU/ml, and E. coliK12 with a detection limit of 10⁶ cells/ml. A flow injection immunoanalysis (FIA) system was able to detect 5×10⁷ CFU/ml of E. coli in artificially contaminated food. An electro-chemiluminescent biosensor was developed for detecting bacteria (Bacillus anthracis, Bacillus subtilis, S. typhimurium, E. coliO157:H7 and Yersinia pestis) from 5×10⁴ to 10⁷ cells/ml. A compact fiber-optic evanscent-wave sensing system was constructed for detection of 10⁴ CFU/ml S. typhimurium. An integrated optic interferometer system was developed for detection of S. typhimurium (heat-treated or boiled) in the range of 1×10⁵ to 1×10⁷ CFU/ml. An electrochemical biosensor was developed to detect Salmonella in food products with detection limit of 10⁵ CFU/ml. The surface plasmon resonance (SPR) biosensor was developed to detect S. enteritidis and L. monocytogenes at the concentration of 10⁶ cell/ml based on prism excitation of surface plasmon and spectral interrogation, Staphylococcal enterotoxin B (SEB) at 5 ng/ml in pure samples, and about 5×10⁷ CFU/ml of E. coliO157:H7. An optical biosensor by using a fourth-generation hydroxy-terminated polyamidoamine dendrimer and SYTOX Green fluorescent nucleic acid stain as a part of the sensing film was applied to detect live Pseudomons aeruginosa. A dual channel surface acoustic wave device was utilized to detect two different bacteria, Legionella and E. coli. An electrochemical biosensor array detected six microbial species including E. coli. A light addressable potentiometric sensor (LAPS) could detect 10³ to 10⁴ CFU/ml of cultured E. coliO157:H7 in PBS solutions. After a 5 to 6-h enrichment at 37° C., E. coliO157:H7 in beef hamburger could also be detected. The quartz crystal microbalance (QCM) was used to detect Shiga-like toxin genes in E. coliO157:H7, 1×10⁷ cells/ml of L. monocytogenes cells in solution and the E. coli heat-labile enterotoxin (LT) and ganglioside GM1. An electrochemical hybridization biosensor was developed for the detection of short DNA fragments specific to the deadly waterborne pathogen Cryptosporidium and the E. coli pathogen. An ion-channel biosensor based on supported bilayer lipid membrane was designed to detect Campylobacter species. An optical biosensor utilized the evanescent field technique for monitoring Staphylococcus aureus and Streptococcus pneumonia in hospital environment. An impedance-based, fieldable biosensor system was extended to detect the foodborne pathogens, E. coli O157:H7 and Salmonella spp. An electrochemical biosensor was designed to detect E. coli toxin with detect limit of 3×10⁻⁶ g/ml. A fluorescence-based biosensor was developed for simultaneous analysis of multiple samples for multiple biohazardous agents. Their limits of detection were achieved in the mid-ng/ml range (toxins and toxoids) and in the 10³-10⁶ CFU/ml range (bacterial analytes). A microfabricated biochip having electrode-containing cavities was prepared to detect viable Listeria innocua. The detection limit was between 1-50 cells in a 5.3 nl (10⁶ CFU/ml). A magnetic focusing immunosensor was invented for the detection of pathogens comprising a laser, an exciting fiber, a collecting fiber, a fiber optic magnetic probe in communication with the collecting and exciting fibers and means for detecting, collecting and measuring fluorescent signals in communication with the collecting fiber. One biosensor system based on the antibody coated magnetic beads and an optical detector was used for the detection of pathogens. The biosensor system could detect Salmonella in inoculated chicken carcass wash water from 2.2×10⁴ to 2.2×10⁶ CFU/ml in 2 hour and E. coliO157:H7 in inoculated ground beef, chicken carcass and romaine samples from 10² to 10⁵ CFU/ml in 1.5 h without any enrichment. A biosensor based on a membrane separator/bioreactor and a UV-Vis detector could detect E. coliO157:H7 from 5.0×10⁴ to 2.2×10⁶ CFU/ml in 50 min without any enrichment. A biosensor consisting of an antibody-immobilized capillary column as a bioseparator/bioreactor with a UV-Vis detector displayed excellent performance. It could detect E. coliO157:H7 from 5.0×10² to 5.0×10⁶ CFU/ml in 1.5 hours without any enrichment. This was ascribed to the large surface area and small diameter of the capillary column that enhances the area for immobilization of antibody and increases the reaction chance between antibodies and antigens.

### SUMMARY OF THE INVENTION

The present invention includes an innovative, sensitive, rapid, portable, and inexpensive biosensor for detection of pathogens. Current state-of-the-art biosensors for detection of pathogens have a number of limitations such as poor detection limit and long assay time. Based on these concerns, the inventors develop a mesoporous-chip based biosensor device that is able to detect very low-level pathogens in a relatively short time. By way of example, and not limitation, a biosensor according to the present invention comprises a mesoporous chip as both bioseparator for separation of pathogens when sample goes through the chip pores and bioreactor for enzyme reaction with labeled antibodies and/or “trapped” biological agents, an immobilized antibody probe within porous microchannel to trap targeted biological agents, an reaction enzyme to release easily detected species, an light source for either absorption or fluorescence measurement, and a pump for sample and reaction agents delivery. This biosensor device is designed in a way that significantly increases the reaction area, and constructed by immobilizing antibodies onto a mesoporous chip surface. The inventors take advantages of the extremely high surface area of mesoporous materials to develop highly sensitive biosensors based on a small chip as a matrix for antibody immobilization and enzyme reaction. In one aspect of the present invention, a mesoporous chip-based sensor for biological agent sensing includes a housing or a chamber, or a flow cell that is established therein. In this aspect, the chamber/cell has a small volume that can hold a mesoporous chip. Moreover, antibody probes are immobilized on the inner surface of the mesoporous microchannel wall. In this aspect of the present invention, sandwich complexes (antibodies immobilized onto the wall of the channel-bacteria-antibodies labeled with enzymes) are formed on the wall of microchannels within the mesoporous chip when the enzyme-labeled antibodies go through the above chip. The labeled enzymes on the sandwich complexes in the microchannels catalyze the enzyme amplification reaction.

In another aspect of the present invention, a mesoporous chip-based sensor for biological agent sensing includes a housing or a chamber, or a flow cell that is established therein. In this aspect, the chamber/cell has a small volume that can hold a mesoporous chip. Moreover, these mesoporous microchannels are in fluid communication with the house chamber as well as the delivery pump, so that samples, antibody solution, buffer, and reaction agents can be delivered through the pump. In this aspect of the present invention, a sample tube is in fluid communication with the house chamber.

In still another aspect of the present invention, a mesoporous chip-based sensor for biological agent sensing includes a housing or a chamber, or a flow cell that is established therein. In this aspect, a light source is used for absorption or fluorescence measurement of reactant/produced chemicals within the microchannel in mesoporous chip. Moreover, a spectrometer is optically connected to the house chamber.

According to a further aspect of the invention, a collimating lens that is adjacent to the house chamber. An optical fiber is installed adjacent to the collimating lens. Accordingly, light source beams passing through optical fibers or directly focusing onto the mesoporous chip and then the collimating lens is used to collect either transmitted lights or fluorescence and focuses these optical beams into another optical fiber. The optical fiber transmits the optical beam to a spectrometer.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The accompanying drawings, which are incorporated in and form a part of the specification, illustrate the embodiments of the present invention and, together with the description, serve to explain the principles of the invention. In the drawings:

FIG. 1 is a typical embodiment of the biosensor system.

FIG. 2 is the details of a mesoporous chip and a house chamber.

FIG. 3 is an illustration of the antibody immobilized mesoporous chip and bioseparator/bioreactor.

### DETAILED DESCRIPTION

Mesoporous materials are a series of nanomaterials developed in the past years. The nanometer-sized to micrometer-sized and regularly arranged pores make these materials very attractive for various applications. Up to date, there are many well-established methods for the fabrication of mesoporous materials in inexpensive manner. The preparation of porous silica has greatly expanded the possibilities for the design of open-pore structures. The microchannels connect the upper and lower faces of the chip in such a manner that fluid can flow through the chip. The mesoporous materials provide not only extremely high surface area, but also good chemical and mechanical durability. Table 1 shows some parameters of a typical porous material. For comparison, the parameters of a capillary column are also provided in the Table. Clearly, the surface area of a small mesoporous chip can be several orders of magnitude higher than a glass capillary column while the depth of the chip is only about 2.5% of the capillary column. Therefore, the inventors here take advantages of the extremely high surface area of mesoporous materials to develop highly sensitive biosensors based on a small chip as a matrix for antibody immobilization and enzyme reaction. With mesoporous materials, the sensitivity for detection of bacteria is significantly enhanced through high reaction/surface area. In this innovative sensor design, first, the antibodies are covalently immobilized onto the wall of the microchannels within the mesoporous chip, and the microchannels containing the immobilized antibody are used as a bioseparator to separate bacteria from the sample when the sample goes through the chip; then, sandwich complexes (antibodies immobilized onto the wall of the channel-bacteria-antibodies labeled with enzymes) are formed on the wall of microchannels within the mesoporous chip when the enzyme labeled antibodies go through the above chip, and the microchannels containing the sandwich complexes are served as a bioreactor because the labeled enzymes on the sandwich complexes in the microchannels catalyze the enzyme amplification reaction; and finally, the pathogens can be detected by measuring the absorbance or fluorescence of the enzyme reaction product when a substrate solution goes through the chip. The advantages of developing microarrays on organized porous materials include: (i) improving responsiveness and dynamic range due to the increased surface area, (ii) reducing assay time due to enhanced mass transport within the channels, (iii) more uniform probe deposition and higher array densities due to improved wetting properties of microporous materials, (iv) enhanced capability, improved detection limits, and minimized sensor device.

TABLE 1
Parameters of various materials

| | Capillary | | |
|---|---|---|---|
| | Column (0.1 × | Single Channel in | Mesoporous Chip |
| Parameters | 200 mm) | Mesoporous Chip | (12 × 12 × 5 mm) |
| Diameter (μm) | 100 | 100 | 100 |
| Channel depth | 200 | 5 | 5 |
| (mm) | | | |
| Channel density | | | 70 channels/mm² |
| Volume (μl) | 15 | 0.039 | 390 |
| Surface Area (m²) | 6.0 × 10⁻⁵ | 1.57 × 10⁻⁶ | 1.57 × 10⁻² |

Responsiveness and dynamic range are factors that are commonly associated with detector performance. In terms of an assay, the responsiveness is given by the slope of the derived signal versus the analyte concentration curve, and the dynamic range is the analytical signal range over which the response curve is linear.

The amount and distribution of antibodies are the primary determining factors for assay responsiveness and dynamic range. The larger the amount of immobilized antibodies, the greater the responsiveness to bacteria, the more signal per unit concentration, and the higher the binding capacity for bacteria providing that the concentrations of the immobilized antibodies are not so high as to prevent immunoreaction by steric hindrance. The amount of antibodies that can be immobilized in a given region is a function of the wall surface area of the microchannels within the chip. The distribution of antibodies is also important because the ability of bacteria to bind to antibodies is a function of the antibody surface density. The mesoporous chip can provide more surface area for antibody immobilization by adding “depth” to the chip, channel density and expanding the lateral dimensions. The microassay signal is eventually detected by measuring the optical absorbance or fluorescence produced from the enzyme amplification catalyzed by the labeled enzyme of the sandwich complexes on the wall of microchannels. Therefore, the design of this innovative sensor device will significantly enhance responsiveness and dynamic range for bacteria detection. The enhancement in responsiveness and dynamic range should be proportional to the ratio of the surface areas of the chip.

In the conventional biosensor design, the mass transport of pathogens to the reaction wall/surface is inefficient because the reaction volume and the space region in which the targeted bacteria exist, are quite large. In our mesoporous geometry, targets are in close proximity to the probes immobilized on the walls inside the microchannels when the target solution flows through microchannels in the chip. The probe and target are physically confined to a small volume. As a result, the rates and efficiencies of immunoreaction between antibodies and bacteria, and enzyme amplification reaction between labeled enzymes and their substrate molecules are greatly enhanced.

The microchannels in the chip can be considered as micro-capillaries; thus, antibody solution is drawn into the void volume of the chip by capillary action. Two distinct advantages of the mesoporous chip result from this property: (i) antibody immobilization is facilitated due to slower evaporation of small (nanoliter) droplets of the probe-deposition solution, and (ii) higher spot density is possible because the same volume of antibody solution has a smaller footprint on the array chip than on a flat matrix due to reduced spreading on the surface. With regard to the former advantage, rapid evaporation of the probe-deposition solution may results in inconsistent immobilization of the antibody on the surface of the microchannels when chemical cross-linking is used. Dehydration prevents the reaction of the cross-linking reagent with the surface wall. In this point of view, this innovative design in the sensor device is more efficiently to use the expensive antibody during immobilization process, and as a result, significantly reduces the cost for the detection.

Based on the above considerations and discussions, the invented biosensor possesses a series of advantages over conventional available devices, such as superior sensitivity and dynamic range due to the increased surface area for antibody immobilization and reaction, sensitive enzyme labels for optical signal amplification, a minimized device due to the small chip and simplified detection scheme, and an inexpensive feature due to the overall simplicity.

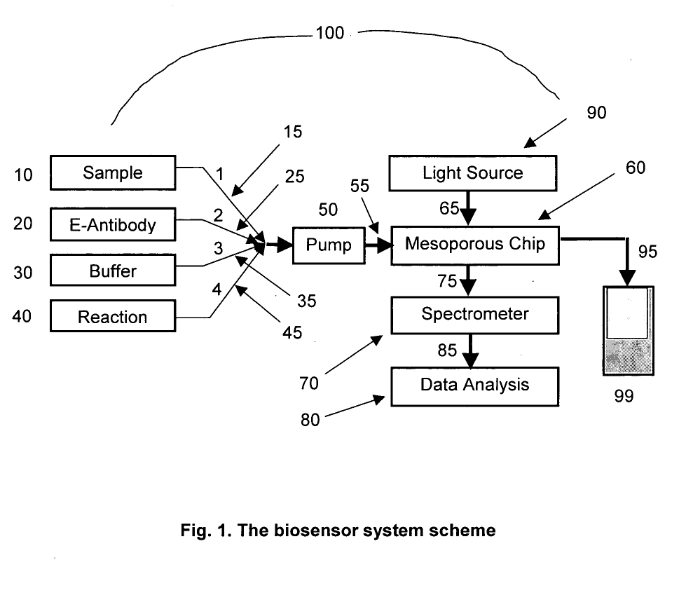

Several embodiments with various spectroscopy methods can be used for bacteria and pathogen detection. Referring more specifically to the drawings, for illustrative purposes the present invention is embodied in the apparatus generally shown in FIG. 1 through FIG. 4. It will be appreciated that the apparatus may vary as to configuration and as to details of the parts, and that the method may vary as to the specific steps and sequence, without departing from the basic concepts as disclosed herein.

Referring initially to FIG. 1, a mesoporous chip based biological sensor for sensing biological agents according to the present invention is shown and is generally designated 100. FIG. 1 shows that the biosensor includes a mesoporous chip 60, an optical light source 90, a spectrometer 70, a data analysis system 80, a sampling pump 50, and various samples and agents, such as sample 10, enzyme-labeled antibody solution 20, buffer solution 30, and reaction solution 40. Sample solution 10 is first delivered by sampling pump 50 through capillary tubes 15 and 55 into the mesoporous chip 60. After the sample delivery, enzyme-labeled antibody solution is delivered in the same way as the solution sample, except through capillary tubes 25 and 55. Similar process is followed for buffer solution through capillary tubes 35 and 55. The final step of delivering reaction solution is then follow up through channels 45 and 55. In this way, one circle of sampling process is finished. Continuous analysis is processed with repeating these sampling and pumping processes with an order of 1, 2, 3, 4, as indicated in FIG. 1.

As shown in FIG. 1, samples delivered into the mesoporous chip 60 are measured with an optical light source 90 where the optical beam 65 passing through the mesoporous chip 60 to reach the spectrometer 70. The spectrometer 70 is used to receive the optical beam 75 and disperse the wavelengths if necessary and then detect intensity of the optical beam as responded signals. A data analysis device 80 is used for data processing and storage. The waste drain from the mesoporous chip flows into a waste container 99 through a channel 95.

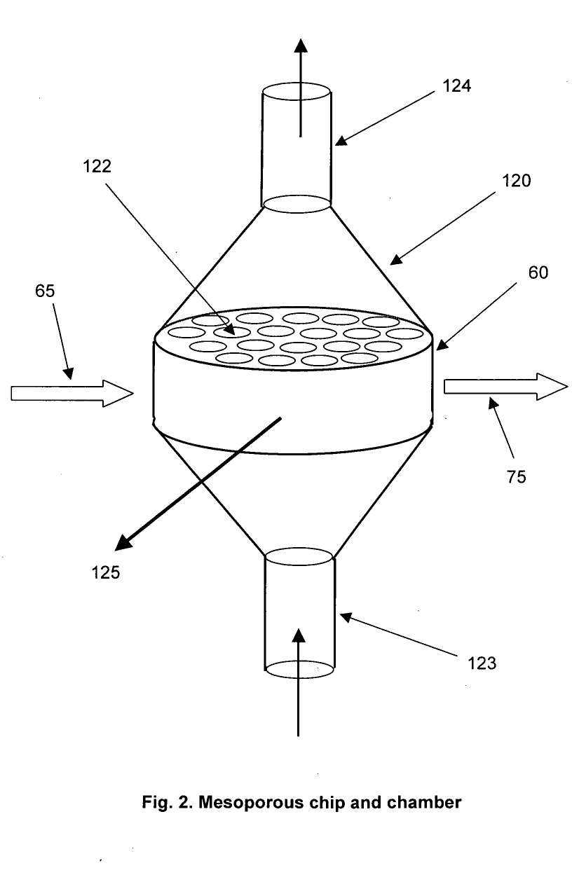

FIG. 2 shows the details of the mesoporous chip 60 and its housing/chamber 120 designate in a preferred embodiment. The mesoporous channels 122 are used to pass various solutions for optical spectroscopy measurement. Sample delivered by sampling pump passes through the channel 123 and then reach the mesoporous chip 60. After reaction within the mesoporous chip, the waste solution is passed to drain through channel 124. The optical measurement can be carried out in at least two typical ways. In the absorption measurement, the light source beam 65 passes through the mesoporous chip 60 and the passed light 75 is measured by using the spectrometer 70 in FIG. 1. In fluorescence measurement, the light source beam 65 is used to excite fluorescence and the produced fluorescence is measured generally in an angle to the excitation light source beam 125, for example, in a right angle, which can minimize the background influence generated by the excitation source.

Referring to FIG. 2, the mesoporous chip generally in a cylindrical shape for easy machine purpose, but it can be in any shape preferably in a narrow rectangle shape, which gives better utilization of the mesoporous chip. The material of mesoporous chip generally selected from transparent or optically transparent materials such as silica, glass or polymers. Other materials with suitability of optical measurement may also be used as substrates.

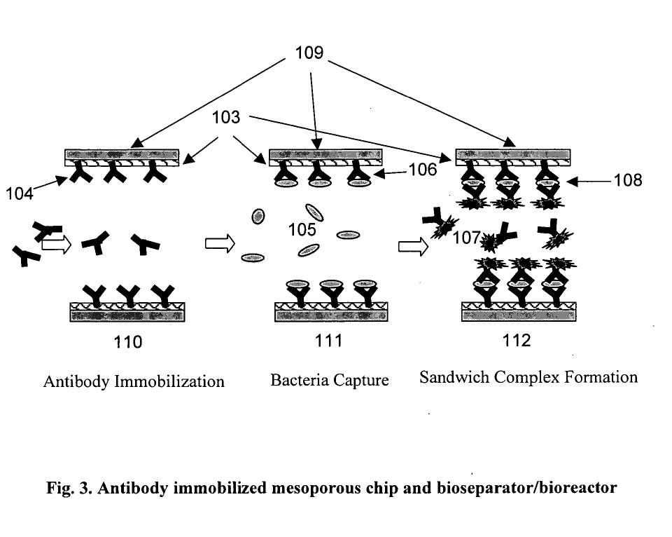

Referring to FIG. 3, a microchannel 109 is used to first immobilize the antibody 104 on the inner surface 103 of the channel wall. Biological agent molecules 105 in sample solution then pass through the microchannel and react with the immobilized antibody 104 on the inner wall 103 to form an antibody-antigen complex 106 on the inner surface of the microchannel 109. When another enzyme-labeled antibody 107 solution passes through the microchannel, an sandwich type complex 108 (antibody immobilized onto the wall of the channel—biological agent—antibody labeled with enzyme) is formed on the inner surface of the microchannel. In this way, when reaction solution is arrived, the enzyme can catalyst the reaction and generate species that are easy for optical detection.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skills in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

1. An apparatus for detecting pathogens or other biological agents, comprising:
a. a housing;
b. a mesoporous chip;
c. a sampling pump;
d. an optical light source;
e. a spectrometer;
f. a data analysis system;
g. a sampling tube or tubes in fluid communication with the said sampling pump.

2. An apparatus as cited in claim 1, wherein the said mesoporous chip is used as a bioseparator to separate biological agents from sample solutions.

3. An apparatus as cited in claim 1, wherein the said mesoporous chip is used as a bioreactor where enzyme catalyst reaction occurred.

4. An apparatus as cited in claim 1, wherein the said mesoporous chip has multiple microchannels, which are in fluid communication with the house chamber.

5. An apparatus as cited in claim 4, wherein the said multiple microchannels are immobilized with antibodies or other agents to trap antigens.

6. An apparatus as cited in claim 1, wherein the said mesoporous chip is made of optically transparent materials.

7. An apparatus as cited in claim 1, wherein the said light source can be any conventional sources or laser sources.

8. An apparatus as cited in claim 1, wherein the said spectrometer can be any spectrometers, monochrometers, prisms, or filters with function of disperse or get rid of interference wavelengths.

9. An apparatus as cited in claim 1, wherein a collimating lens and an optical fiber can be used to focus optical beam onto the mesoporous chip and then collect transmitted lights or fluorescence to a spectrometer.

10. An apparatus as cited in claim 1, wherein the said mesoporous chip is in fluid communication with sampling pump and waste streams.

11. An apparatus as cited in claim 1, wherein the said sampling pump is in fluid communication with several solution/reaction cells including sample cell, enzyme-labeled antibody cell, buffer cell, and reaction solution cell.

12. A sensor for sensing biological agents, comprising:
a. an apparatus having a mesoporous chip with multiple microchannels;
b. a sampling pump in fluid communication with mesoporous chip and sample solutions;
c. a spectrometer optically connected to the mesoporous chip;
d. a light source for spectrometry.

13. A sensor as cited in claim 11, wherein the said apparatus comprises:
a. a housing;
b. a tube or tubes in fluid communication with sampling pump and mesoporous chip;
c. a data analysis system;
d. a waste container.

14. A method for sensing biological agents, comprising:
a. antibody immobilization on the inner surface of the mesoporous chip channels;
b. enzyme-labeled antibody for catalysis of reaction;
c. introducing at least one sample into the mesoporous chip;
d. forming sandwich type complex;
e. through light absorption or fluorescence measurement for agent detection;
f. using a sampling pump to deliver sample solution, enzyme-labeled antibody solution, buffer solution, and reaction solution.
g. detecting a chemical species resulted from the catalyst reaction.

15. A method for sensing biological agents as recited in claim 13, wherein the said antibodies are immobilized on the surface of the inner microchannels through chemical covalent bonding or hydrogen bonding.

16. A method as recited in claim 13, wherein the said enzyme-labeled antibody is used for catalysis of chemical reaction within the microchannels.

17. A method as recited in claim 13, wherein the said sandwich type complex is in a format of “antibody immobilized onto the wall of the channel—biological agent (target molecule)—antibody labeled with enzyme”.

18. A method as recited in claim 13, wherein the said optical measurements can be through absorption.

19. A method as recited in claim 13, wherein the said optical measurements can be through fluorescence.

20. A method as recited in claim 13, wherein the said sampling pump in fluid communication with mesoporous chip and to deliver sample solution, enzyme-labeled antibody solution, buffer solution, and reaction solution.

21. A method as recited in claim 13, wherein the said chemical species resulted from the catalyst reaction is detected for sensing biological agents, for example, hydrogen peroxide.
